(19) United States
(12) Patent Application Publication
(10) Pub. No.: US 2025/0376395 A1
(43) Pub. Date: Dec. 11, 2025

(54) System and Method for Vermiculture Filtration

(71) Applicant: George Hahn, Cardiff, CA (US)

(72) Inventor: George Hahn, Cardiff, CA (US)

(21) Appl. No.: 18/739,154

(22) Filed: Jun. 10, 2024

(51) Int. Cl. C02F 3/32; A01K 67/033; C02F 9/00; C02F 101/16; C02F 103/00; C02F 103/20; C05F 17/05; C05F 17/80

(52) U.S. Cl. CPC C02F 3/327; A01K 67/33; C02F 9/00; C05F 17/05; C05F 17/80; C02F 2101/163; C02F 2103/005; C02F 2103/20; C02F 2201/32; C02F 2203/006; C02F 2209/44; C02F 2301/08; C02F 2303/02; C02F 2303/04

(57) ABSTRACT

A system and method including means for holding waste material including wastewater, contaminated liquid, animal waste, human waste or sludge; means for housing earthworms; means for transferring the wastewater from said means for holding wastewater to said means for housing earthworms; means for timing the transfer of the wastewater from said means for holding wastewater to said means for housing earthworms to allow earthworms in said earthworms housing means to process the waste water; means for removing suspended matter/debris and/or floating/sinkable particles present in the wastewater; means for draining the processed wastewater to said means for removing suspended matter/debris and/or floating/sinkable particles present in the wastewater; means for sterilizing the wastewater with UV light; and means for draining the processed and polished waste water to said means for sterilizing the wastewater with UV light.

### CROSS-REFERENCE TO RELATED APPLICATIONS

Not applicable.

### RELATED CO-PENDING U.S. PATENT APPLICATIONS

Not applicable.

### INCORPORATION BY REFERENCE OF SEQUENCE LISTING PROVIDED AS A TEXT FILE

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to techniques for treating contaminated water. More particularly, certain embodiments of the invention relate to a vermiculture filtration system.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

A common byproduct of many farms that practice animal husbandry is the waste that their animals produce. Animal waste is typically a source of major pollution and may give off an unnecessary amount of greenhouse gasses, manure odors, contain various strains of bacteria and nitrates, and contaminate ground water if not carefully handled. Pits or sludge ponds are typically used to contain animal waste, and various techniques may be used to process the animal waste depending on the needs of the farm.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that Earthworms have been known for several hundred years to have the ability to convert various manure and waste streams into rich soil amendments that replenish soil to make the soil biologically healthy. Earthworm digestive systems may be used to process contaminated liquid by creating an environment where the earthworms can drink the water to filter out the solids and produce earthworm castings. Earthworm farms may be used to process animal waste and thus reduce the amount of pollution that farms practicing animal husbandry produce. Such earthworm farms are typically managed outdoors and may be practiced in areas with an environmental temperature that can support the earthworms. Temperature can have a great effect on the productivity of earthworm farms, as earthworms may slow down reproduction and feeding while outside an optimal zone and may even die if temperatures get too extreme.

In view of the foregoing, it is clear that these traditional techniques are not perfect and leave room for more optimal approaches.

### BRIEF DESCRIPTION OF THE DRA WINGS

The present invention is illustrated by way of example, and not by way of limitation, in the figures of the accompanying drawings and in which like reference numerals refer to similar elements and in which:

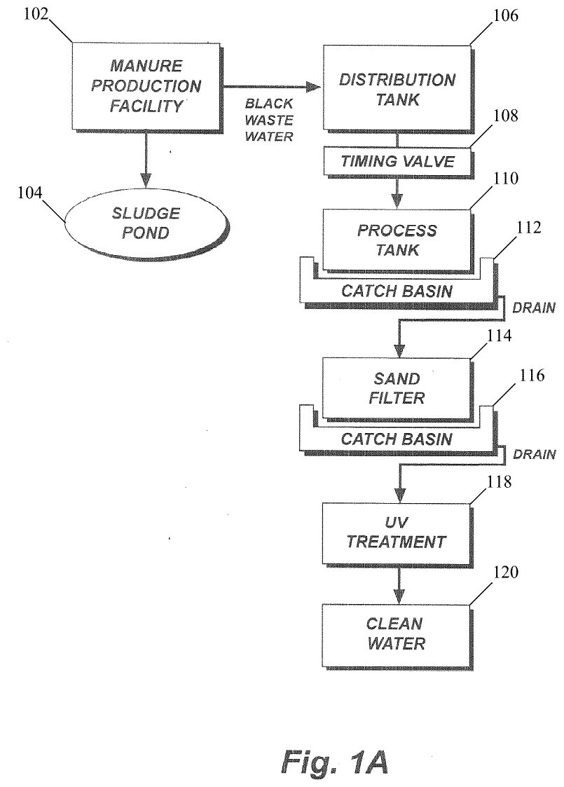

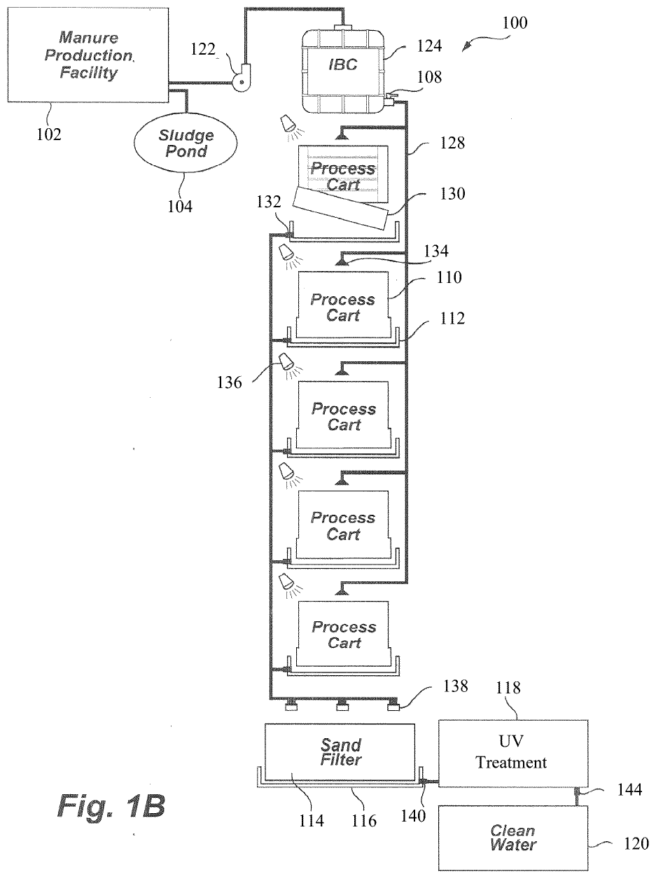

FIGS. 1A-1C illustrate an exemplary Vermiculture Filtration System (VFS) in accordance with an embodiment of the present invention, wherein FIG. 1A depicts a flow diagram from a manure production source to finished castings and clean water produced by a VFS, FIG. 1B illustrates the stacked gravity flow of the VFS from the containment tank to clean water, and FIG. 1C illustrates the stacked gravity flow of a VFS placed on pallet racks;

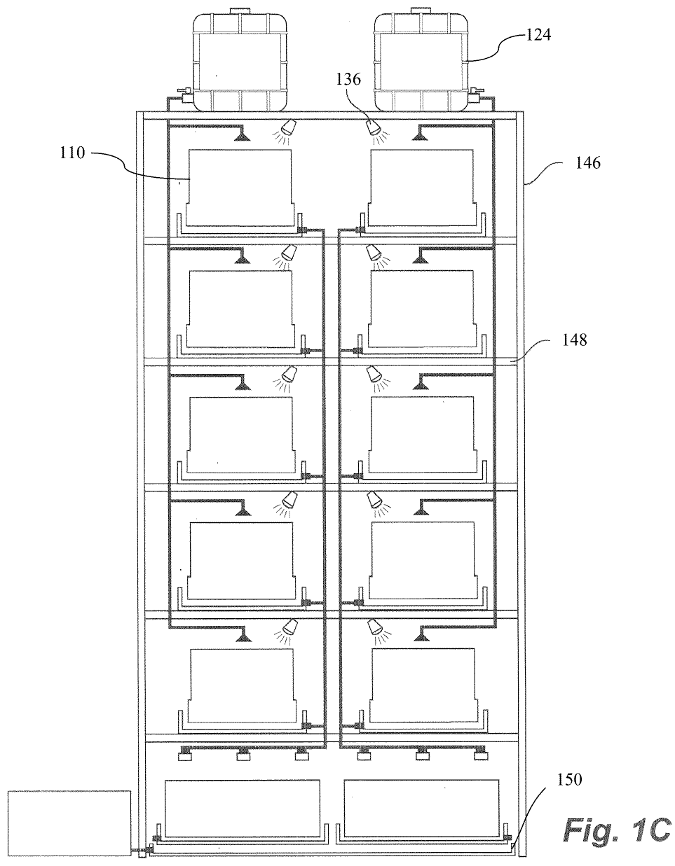

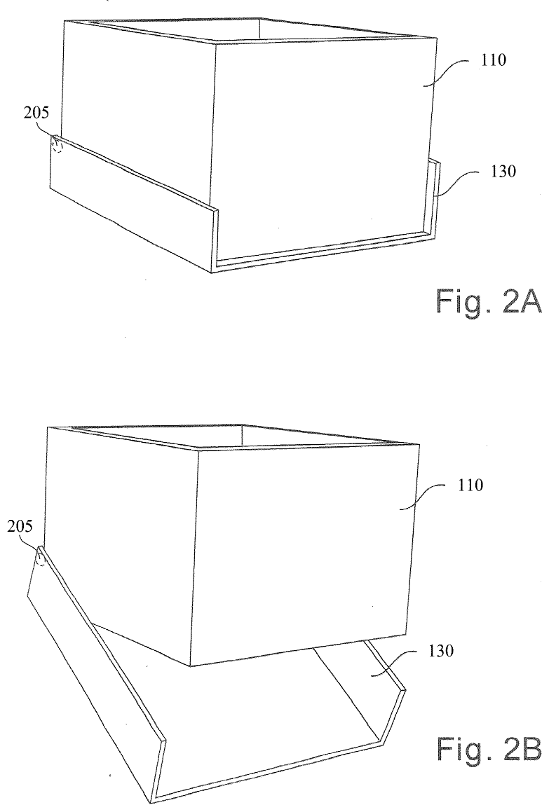

FIGS. 2A-2B illustrate an exemplary VFS process tank with a hinged dump bottom, in accordance with an embodiment of the present invention;

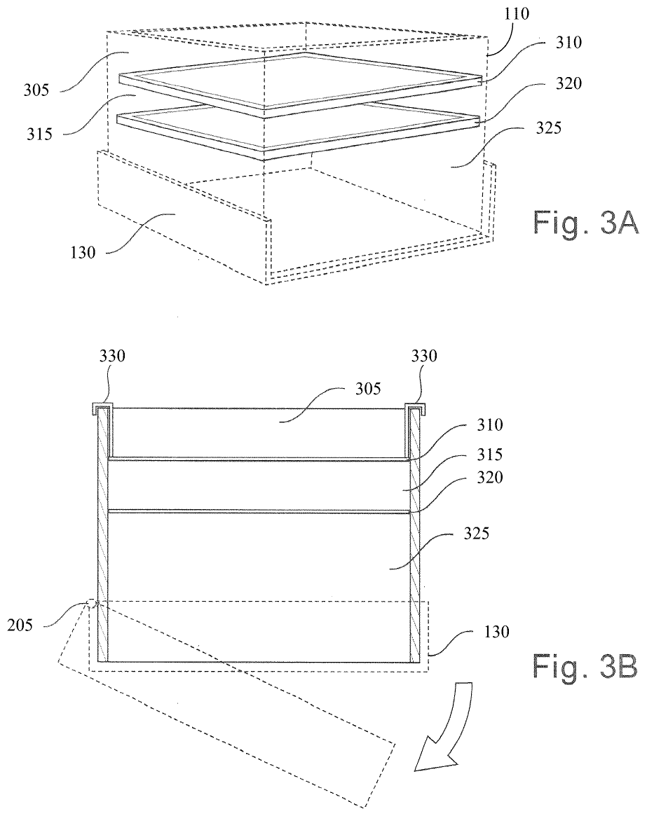

FIGS. 3A-3B illustrate an exemplary VFS process tank with egg capsule juvenile separation screen and adult earthworm separation screen, in accordance with an embodiment of the present invention;

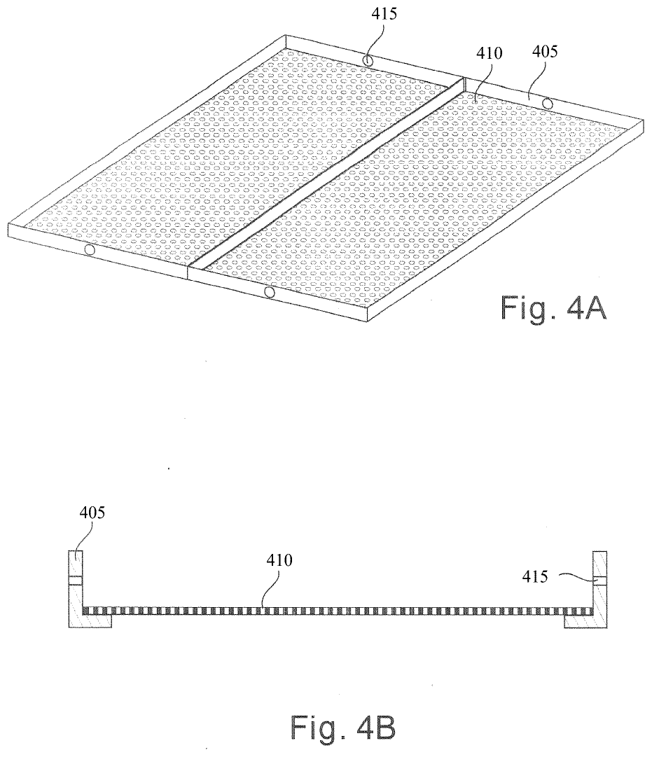

FIGS. 4A-4B illustrate exemplary separation screens, in accordance with an embodiment of the present invention;

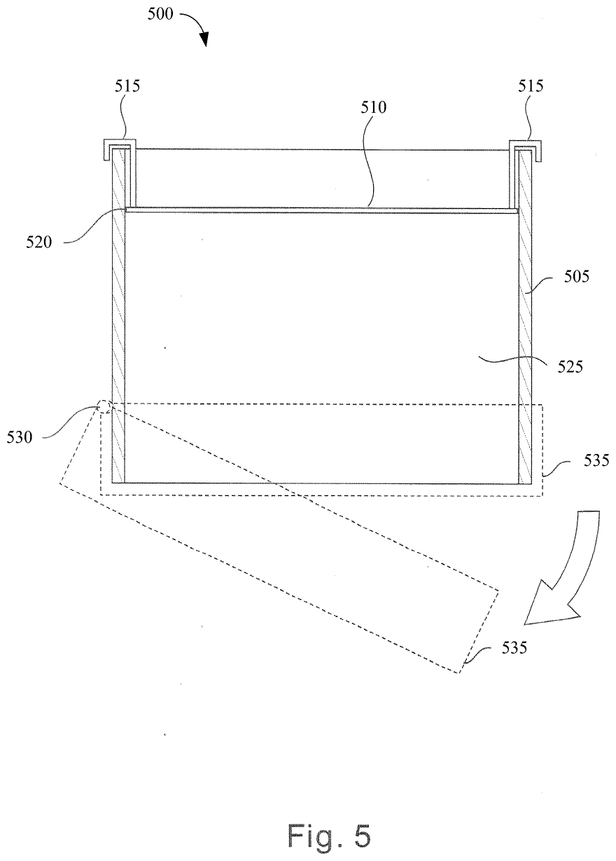

FIG. 5 illustrates an exemplary sand filter with clean-up screen in accordance with an embodiment of the present invention;

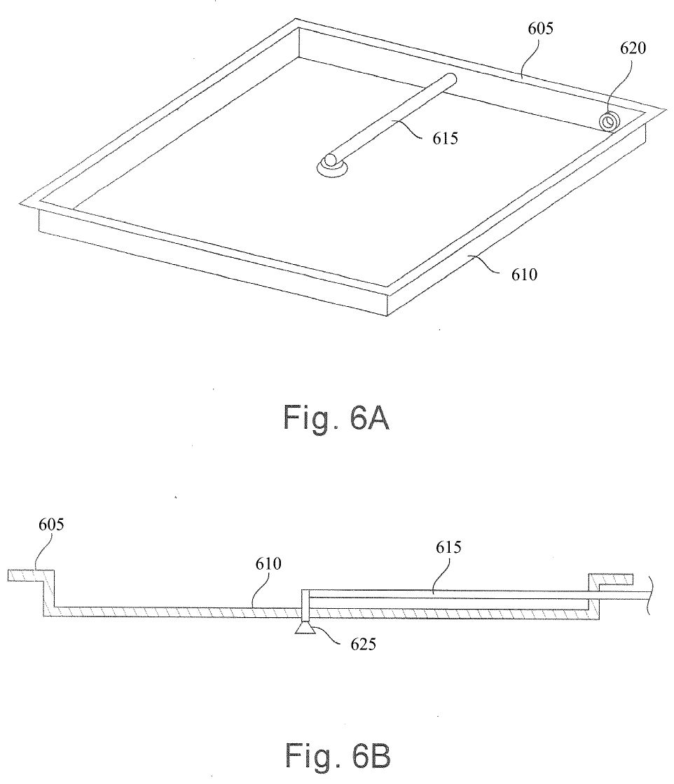

FIGS. 6A-6B illustrate an exemplary catch basin in accordance with an embodiment of the present invention; and

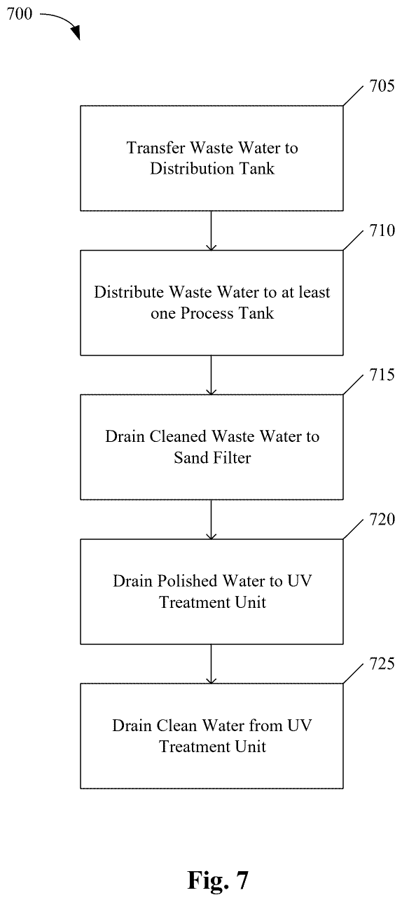

FIG. 7 illustrates a flow chart of an exemplary process for using VFS, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications described herein, as these may vary. It is also to be understood that the terminology used herein is used for the purpose of describing particular embodiments only and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial,” “nearly,” “almost,” “about,” “generally,” “largely”, “essentially,” “closely approximate”, etc.

As will be established in some detail below, it is well settled law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to, nearly, almost, about,” connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [” substantially “] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognized in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of Claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar to or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” include the stated particular feature, structure, or characteristic.

References to “user,” or any similar term, as used herein, may mean a human or non-human user thereof. Moreover, “user,” or any similar term, as used herein, unless expressly stipulated otherwise, is contemplated to mean users at any stage of the usage process, to include, without limitation, direct user(s), intermediate user(s), indirect user(s), and end user(s). The meaning of “user,” or any similar term, as used herein, should not be otherwise inferred or induced by any pattern(s) of description, embodiments, examples, or referenced prior art that may (or may not) be provided in the present patent.

References to “end user,” or any similar term, as used herein, is generally intended to mean late-stage user(s) as opposed to early-stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising” And “contain” and variations of them-Such terms are open-ended and mean “including but not limited to.” When employed in the appended claims, this term does not foreclose additional structure or steps. Consider a claim that recites: “A memory controller comprising a system cache . . . ” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

All terms of exemplary language (e.g., including, without limitation, “such as”, “like”, “for example”, “for instance”, “similar to”, etc.) are not exclusive of any other, potentially, unrelated, types of examples; thus, implicitly mean “by way of example, and not limitation . . . “, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phrase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the disclosed and claimed subject matter may include the use of either of the other two terms. Thus, in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Moreover, any claim limitation phrased in functional limitation terms covered by 35 USC § 112(6) (post AIA 112(f)) which has a preamble invoking the closed terms “consisting of,” or “consisting essentially of,” should be understood to mean that the corresponding structure(s) disclosed herein define the exact metes and bounds of what the so claimed invention embodiment(s) consists of, or consisting essentially of, to the exclusion of any other elements which do not materially affect the intended purpose of the so claimed embodiment(s).

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

Some embodiments of the present invention and variations thereof, relate to method and system for filtration of waste materials including, not a limitation, a combination of liquid and solid such as animal manures and other organic contaminated liquids (such as human bio-solids) using earthworms to significantly reduce greenhouse gas production, eliminate odor emissions, reduce nitrate contamination, and eliminate human indicator pathogens while producing organic soil amendment and usable water for use in agriculture and for reuse as wash down water. System is called “Vermiculture Filtration System” (VFS).

In one embodiment of the present invention, a system for changing animal manures (e.g., feces, urine, and wash down water-manure water) by using earthworm digestion in a controlled and contained process is disclosed. The system may include, not a limitation, one or more containment and/or distribution tanks to hold and feed the contaminated liquid or wastewater to contain the odor production from the contaminated liquids before entering the process tanks. The liquid is then distributed through an opening from the containment tank and gravity fed into a processing tank. The processing tank may include a layer of various species of earthworm through which the liquid is filtered by the digestion of the earthworms. The earthworms may remove the solids from the contaminated liquid producing earthworm castings. Earthworm castings are an organic form of fertilizer produced from earthworms. Also known as vermicast, earthworm castings is essentially earthworm waste or manure, otherwise known as worm poo. As earthworms eat through the contaminated liquid or wastewater, their waste creates an optimal soil enricher. The earthworm castings produced from contaminated liquid or wastewater may provide the same benefit as dry environment earthworm castings. But changing the system so the earthworms may function to process a contaminated liquid that provides an added benefit of stopping the production of GHG and odors that are caused from the anaerobic degradation of the solids. The earthworm castings may be formed when the earthworm removes the solids from the liquid. The castings produced are aerobic in nature which stops the GHG & noxious anaerobic odor production.

The earthworms may also remove chemical and biological contaminants from the liquid. The filtered and decontaminated liquid passes through the process tank into a sand filter tank. The liquid may be processed through a UV light treatment sterilization to provide clean pure water that may be safely used for agriculture irrigation and wash down water.

The removal of the solids and biological & chemical contaminants by the earthworms producing earthworm castings may eliminate the solids from the waste stream that would normally produce greenhouse gases (GHG). Academic testing has shown the elimination of the GHG production is over 90%. The earthworm digestion removal of the contaminants may eliminate the source products that produce the objectionable animal manure odors.

In some embodiments, the individual process containers are designed to separate the mature earthworms and separate egg capsules and juvenile earthworms from processed earthworm castings for reuse without using a mechanical screen. This allows the earthworm castings to then be removed from the process containers to be made available for various agriculture applications. The castings now produce virtually no GHG and no noxious odors. The process container design includes a hinged bottom for simplified removal of the castings. The containment tanks, process tanks, and sand filter tanks are designed to be held in standard pallet storage rack system to allow for gravity fed processing, ease of removal, contaminated liquid distribution, cleaned liquid capture, and castings removal. The holding racks are designed as metal wire mesh to allow the cleaned liquids to pass through to a catch basin held under the wire mesh rack. The catch basin drains to the sand filter. Holds the piping to distribute the manure water to a process tank held below the catch basin and holds the containment light to keep the earthworms inside the process container. Since the earthworms are sensitive to light, a light is installed over each process tank to keep the earthworms inside the tank even when the tank is filled near the top. This complete filtration process is designed for operation inside an enclosed building to provide environmental control of temperature, rain, snow, and other airborne products. The building design may also include, not a limitation, final air filtration to further mitigate all noxious odors.

In other embodiments, the disclosed operational process is designed for multiple biological waste applications to use multiple types of earthworms to digest biological wastes to clean and filter animal manures (dairy, beef, swine, chicken, etc.), human wastes, food industry, and agricultural processing wastes. The earthworms housed in a concentration of more than two pounds/square foot are able to filter waste liquids by removing the solids from the liquid. The solids may be transformed by the digestion process of the earthworms to produce clear water in about two (2) hours. By supplying the waste liquid in a pulse time feeding sequence, the earthworms are able to filter any biological waste water. The clear water is then polished using a sand filter then processed using a UV sanitizing process. The sanitized water may be used as washdown water which will reduce the water usage of any waste producing facility or irrigation water. Since the system uses multiple distribution, process, and sand filter tanks, the system has virtually a proximate 100% reliability. If one vertical, gravity fed tank system encounters an operational problem, the problem does not stop the full operation of the other tanks.

In one embodiment, the waste water is pumped to a top-level distribution tank equipped with a bottom drain. The plumbing from the distribution tank is designed to feed each of the process tanks a specific volume of waste water in a pulse timing sequence. A control valve from the bottom of the distribution tank will open for a specified time period to feed a measured volume of the waste water through a sprinkler head onto the top of each process tank below the distribution tank. Then the feed is paused allowing time for the earthworms to digest all of the waste water fed into the process tank.

In some embodiments, one or more process tanks may serve several separate functions to filter the waste. A specified thickness of earthworm bedding material may be placed in the bottom of a clean process tank. This bedding material will be a special mix of cellulose material. At least 2 # of earthworms per square foot is loaded into the clean process tank. Two divided screens may be held at the top of the process tank. The top screen set may be a specified distance from the top, roughly 5″-7″ to hold the mature earthworms when the level of the separated solids formed into earthworm castings has filled the process tank to the top. A 2^{nd} set of screens may be held at approximately 10-12″ to hold the egg capsules and juvenile earthworms. The exact height may depend on the waste treated and the earthworm variety mix. The bottom of the process tank may be hinged at one end to allow the cleaned water to drain from one the end opposite the hinge point. The hinged bottom may allow a rapid and easy removal of the castings from the process tank. When the tank is filled, the mature worms will be found in the bedding mix at the top and can be easily removed by lifting out the top screen. The top screen may be used to remove the mature earthworms without the necessity of putting the process tank mix through a trommel screen which damages the earthworms. The screen is divided in half so the worm population can be divided in half. Pivot holes are located in each half to allow the screen to be turned over and deposit in the bottom of each process tank. The lower level will hold the egg capsules and juvenile worms.

As the earthworms remove the biological solids from the waste water, the solids may be transformed into earthworm castings. At this transformation, the earthworm castings are now stable, aerobic, and no longer produce greenhouse gasses (GHG). The earthworm transformation removes roughly 98% methane, 96% ammonia, 92% carbon dioxide, and 92% Nox. The transformation process may eliminate the production of noxious odors which emanate from the waste water liquid without filtration by the earthworm digestion process.

The cleaned waste water is gravity fed to a sand filter to polish the water. The same process tank is used as the sand filter tank so the water can drain out one side and the sand may be easily dumped and refilled. A screen is located at about 6″ below the top for easy removal of the top six (6″) inches as this layer of sand gets plugged. The proximate 6″ layer of sand may be placed into a process tank. The sand filter may polish the water making the water clearer. The sand filter polished water is then fed through a UV sterilization unit to assure all pathogens are removed. Now the water can be reused as washdown water, irrigation water, or put into a clean water stream. The Vermiculture filtration process may be located inside an insulated building where the proper temperature may be maintained for optimal processing at around 65 F-80 F. The building may be equipped with a filtration grid that may be filled with activated carbon or pelletized earthworm castings to remove and nascent biological odors produced from any waste water that did not get processed. The Vermiculture Filtration System provides cleaning of waste water at a very low operational cost. Since the system is gravity fed, the only pumping energy may be that needed to fill the distribution tanks and the UV sterilization. The insulated building requires limited temperature control.

The present invention will now be described in detail with reference to embodiments thereof as illustrated in the accompanying drawings.

FIGS. 1A-1C illustrate an exemplary Vermiculture Filtration System (VFS) in accordance with an embodiment of the present invention, wherein FIG. 1A depicts a flow diagram from a manure production source to finished castings and clean water produced by a VFS, FIG. 1B illustrates the stacked gravity flow of the VFS from the containment tank to clean water, and FIG. 1C illustrates the stacked gravity flow of a VFS placed on pallet racks.

With reference to FIG. 1A, manure production facility 102 may produce black waste water, which may be pumped into containment and/or distribution tank 106 and, optionally, in the case that distribution tank is full, sludge pond 104. Manure production facility 102 may be any type of manure production facility, and may produce any type of waste, such as, but not limited to, dairy, beef, swine, chicken, human, food industry, agricultural processing, etc. In many cases, manure production facility 100 may be adapted to use a VFS by directing waste from both sludge pond 104 and manure production facility 100 to distribution tank 106. Black waste water may be pumped or gravity fed.

Pumping to the top of the pallet rack stack may allow the process to be gravity fed. Once the VFS is fully operational, sludge pond 104 may optionally be eliminated. Distribution tank 106 may drain to process tank 110 via timing valve 108. Timing valve 108 may control the volume of black waste water that is distributed to process tank 100. A pulse timing sequence may be utilized by timing valve 108, distributing black waste water to process tank 110 through distribution piping on a timed sequence, allowing earth worms in process tank 110 time to digest black waste water. Distribution piping may be, for example, without limitation, Polyvinyl chloride (PVC) piping, copper piping, steel piping, Stainless Steel, etc. Process tank 110 may contain, not a limitation, earthworms used to clean black waste water.

Cleaned waste water from process tank 110 may be caught via process tank catch basin 112 and drained to sand filter 114. Sand filter 114 may be used to further polish the cleaned waste water. The sand filter is generally used for the removal of suspended matter/debris, and/or floating/sinkable particles present in the waste water. The waste water may flow vertically through, not a limitation, a bed of sand and/or gravel. The floating and/or sinkable particles may be removed by way of absorption an/or physical encapsulation. Sand Filters are common to clean up swimming pool water acting as a fine mesh filter. The polished clean waste water may be caught by sand filter catch basin 116, and subsequently drained to Ultraviolet (UV) treatment 118. UV treatment 118 may use ultraviolet light to treat the polished clean waste water in order to further disinfect the polished clean waste water. As will be appreciated by one skilled in the art, other means of purifying the polished clean waste water may be used such as, without limitation, boiling, using an iodine solution, tablets, or crystals, using chlorine drops, using a water filter, etc. Further, one or more water purification steps may be used to purify cleaned waste water after processing in process tank 110. After the polished clean waste water is further purified by, for example, not a limitation, UV treatment 118, clean water may be collected in a clean water storage tank 120 for distribution.

With reference now to both FIG. 1A and FIG. 1B, VFS 100 may be used to process black waste water from one or more of manure production facility 102 and sludge pond 104. Black waste water may be distributed to intermediate bulk container (IBC) 124 via pump 122. Pumps can be centrifugal or positive displacement. In some embodiments, IBC 124 may be 250 gallons in volume, however, any size IBC may be used. As will be appreciated by one skilled in the art, any type of waste storage container may be used, such as, without limitation, rigid IBC tanks, flexible IBC tanks, etc., and may be composed of any material such as, without limitation, plastic, composite, carbon steel, stainless steel, fiberglass, etc. Black waste water may be distributed to process carts 110 via timing valve 108 as described above. Black waste water may be sprinkled into process carts 110 via distribution nozzle 134 for even distribution of black waste water. Multiple process carts 110 may be used so that if one of process carts 110 encounters an operational problem, the operation of the other tanks may still continue. Earthworms may be used in process carts 110 to process black waste water, removing biological solids from the waste water and transforming the solids into earthworm castings. Each process cart may have hinged dump bottom 130 to easily remove earthworm castings from process carts 110. Light 136 may be placed above each process cart 110 in order to keep earthworms within process carts 110. After black waste water is processed via process carts 110, cleaned wasted water may be caught via process cart catch basin 112 and drained to sand filter 114 via process cart drain 132. Cleaned waste water may be evenly distributed to sand filter 114 via sprinklers or other distribution devices 138. Cleaned waste water may be gravity fed through sand filter 114, polishing the cleaned waste water, and subsequently caught in sand filter catch basin 116. Polished cleaned waste water may be drained from sand filter catch basin 116 to UV treatment 118 via sand filter drain 140. UV treatment may use ultraviolet light to further purify polished cleaned waste water. After treatment in UV treatment 118, clean water 120 may be drained via UV treatment drain 144 and subsequently collected.

With reference to both FIG. 1A and FIG. 1C, VFS 100 may be stored on, for example, pallet rack 146. Pallet rack 146 may have horizontal wire mesh platforms 148, allowing liquid including overflow liquid to pass through to a catch basin 150 below. IBC 124 may be stored on the top rack of pallet rack 146 so that black waste water may be gravitationally fed to process carts 110. Earthworms may be more efficient in processing black waste water while the environmental temperature is within a certain range. Because VFS 100 may be stored on pallet rack 146, VFS 100 may more easily be maintained inside an insulated, temperature-controlled building. In the preferred embodiment, the temperature inside the insulated building may be maintained for optimal processing of waste water, between 65° F.-80° F. It should be appreciated by one skilled in the art that temperatures outside the range of 65° F.-80° F. may be used as well. Additionally, the insulated building may be equipped with a filtration grid that may be filled with activated carbon or pelletized earthworm castings to remove and nascent biological odors produced from any black waste water that did not get processed. While VFS 100 may be used inside an insulated building, it should be appreciated that VHS 100 is not limited to being used inside an insulated building and may be used in an outdoor environment.

FIGS. 2A-2B illustrate an exemplary VFS process tank with a hinged dump bottom, in accordance with an embodiment of the present invention. With reference now to both FIG. 1A and FIGS. 2A-2B, process tank 110 may have hinged dump bottom 130, allowing a user to easily drain cleaned water and remove earthworm castings that are a byproduct of processing black waste water. Process tank 110 may be composed of, for example, without limitation, painted steel, plastic, carbon steel, fiberglass or other materials, As shown in FIG. 2A, hinged dump bottom 130 is shown in base form. As shown in FIG. 2B, hinged dump bottom may rotate about hinge point 205.

FIGS. 3A-3B illustrate an exemplary VFS process tank with egg capsule juvenile separation screen 320 and adult earthworm separation screen 310, in accordance with an embodiment of the present invention. With reference now to FIG. 1B, FIG. 2A, and FIGS. 3A-3B, process tank 110 may be divided into three separate areas: adult area 305, middle area 315, and bottom area 325. Bedding material may be initially placed at the bottom of a clean process tank 110, and, preferably, at least two pounds per square foot of earthworms may be loaded into the clean process tank 110. Top screen set 310 may be a specified distance from the top 5″-7″ to hold the mature earthworms when the level of the separated solids formed into earthworm castings has filled the process tank to the top. Second set of screens 320 may be held at 10-12″ to hold the egg capsules and juvenile earthworms. The exact height will depend on the waste treated and the earthworm variety mix. The bottom of process tank 110 may be hinged at one end to allow the cleaned water to drain from the end opposite hinge point 205. Hinged bottom 130 allows a rapid and easy removal of the castings from process tank 110. When the tank is filled, the mature worms will be found in the bedding mix at top area 305 and can be easily removed by lifting out top screen 310 via hooks 330. Top screen 310 can be used to remove the mature earthworms without the necessity of putting the process tank mix through a trammel screen which may damage the earthworms. Middle area 315 may be used to hold the egg capsules and juvenile worms. The worms follow the food. They easily climb through the holes in the screen when the bottom tank is full. New bedding fills an empty tank up to the level where the screen filled with worms will be set over. As unprocessed black water is pulsed through, the worms drop down to the bedding in the tank. As the level of castings builds up from the bottom, the worms follow the food so stay at the top of the castings. When the level reaches the screens, the worms crawl through the holes. The juveniles are just behind (under). Once at the top of the box, the majority of the worms, juveniles, and unhatched egg capsules will be in the top screens. The worms are then able tom be easily moved to a new box. The screens are split in half so the worms can be divided to start two empty boxes.

FIGS. 4A-4B illustrate an exemplary separation screen, in accordance with an embodiment of the present invention Separation screen 405 may be divided in half so the worm population may be divided in half. Pivot holes 415 may be located in each half of separation screen 405 to allow each half of separation screen 405 to be turned over and deposit in the bottom of each process tank. Separation screen 405 may use holes 410 to separate, for example, adult earthworms from juvenile earthworms and eggs. Holes 410 may be of different sizes depending on which area separation screen 405 is screening for. The hole sizing is merely large enough for the worms to crawl through. The castings agglomerate at this moisture level so they will not fall through the small, worm sized holes. Holes 410 allow the earthworms to pass up through as the process tank fills with castings. Separation screen 405 may use for example, without limitation, aluminum angle for framing and perforated aluminum or stainless steel for the bottom. As will be appreciated by one skilled in the art, other materials may be used to form separation screen 405 such as, without limitation, copper, galvanized steel, plastics, etc. Screen materials can be plastic, stainless, fiberglass. Carbon steel rusts so not used.

FIG. 5 illustrates an exemplary sand filter with a clean-up screen in accordance with an embodiment of the present invention. Cleaned waste water may be gravity fed to sand filter 500 to polish the water. Hinged bottom 535 of sand filter 500 may swing open opposite hinge point 530, allowing water to easily be drained from the side opposite of hinge point 530 and sand may be easily dumped and refilled. Screen 520 may be located 6″ below the top of sand filter tank 505 and supported by hooks 515, separating top layer 510 from bottom area 525. As will be appreciated by one skilled in the art, screen 520 may be any distance from the top of sand filter tank 505 and is not limited strictly to 6″ from the top. Top layer 510 may be separated allowing for easy removal of top layer 510 as the sand in top layer 510 may easily get plugged. The sand in top layer 510 may be placed into a process tank. In commercial sand filters used in swimming pools, the top of the filter plugs first. These filters have a built-in system to reverse the flow and flush out the separated detritus and send down the drain. The VFS system does not have the ability to reverse flow flush the sand, so the top layer needs to be periodically replaced. Sand filter 500 may be used to polish the cleaned waste water making it clearer. Sand is well proven as a filter media to polish water. All of the sand could be periodically replaced as it plugs. By replacing just the top inches, the timing for full bix replacement is extended. The hinged bottom allows for easier dumping.

FIGS. 6A-6B illustrate an exemplary catch basin in accordance with an embodiment of the present invention. Catch basin 605 may be installed under the process tanks and may be used to catch cleaned water on bottom surface 610 and drain to the sand filter via drain 620. Catch basin 605 may be composed of acrylonitrile butadiene styrene (ABS) vacuum formed plastic, and/or any material that can hold water can be used as a catch basin; steel, stainless steel, copper, etc. Because the process tanks may be stacked in a vertical fashion, distribution piping 615 containing black waste water may pass through catch basin 605 and distribute the black waste water to the process tank below via distribution nozzle 625. Although distribution piping 615 is depicted passing through catch basin 605, it should be appreciated by one skilled in the art that distribution piping 615 along with distribution nozzle 625 may also be mounted to the bottom of catch basin 605.

FIG. 7 illustrates a flow chart of an exemplary process for using VFS, in accordance with an embodiment of the present invention. Process 700 begins with a step 705 where waste water may be transferred to a distribution tank. Waste water may be transferred from any source, such as, without limitation, a sludge pond, a manure production facility, etc. Waste water may be transferred by any means, such as, without limitation, using a pump, gravity fed, manually, etc. Process 700 then continues to a step 710 where waste water may be distributed to at least one process tank. Waste water may be distributed to process tanks using a pulse timing sequence to allow enough time for earthworms in process tanks to process the waste water. After the waste water is processed in the process tanks, process 700 then continues to a step 715 where the processed/cleaned waste water may be drained to a sand filter to be polished. Process 700 may then drain the polished water to a UV treatment unit in a step 720 for further purification. Finally, the process may continue to a step 725 where clean water may be drained from the UV treatment unit.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

The key to this invention is to use specialized earthworms to process and clean the black manure water in boxes that can be easily dumped and refilled and can be stacked vertically on pallet racks to more easily facilitate dump and refill. The earthworms remove the manure GHG (methane, ammonia, Nitrous Oxides) by their internal digestion process). The contaminants removed when the earthworms produce castings is why the manure water goes into the VFS boxes and comes out clear.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” or “steps for” claim limitation implies that the broadest initial search on 35 USC § 112(6) (post AIA 112(f)) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC § 112(6) (post AIA 112(f)) when such corresponding structures are not explicitly disclosed in the foregoing patent specification. Therefore, for any invention element(s)/structure(s) corresponding to functional claim limitation(s), in the below claims interpreted under 35 USC § 112(6) (post AIA 112(f)), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC § 112(6) (post AIA 112(f)) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserves its right to later amend the present application to explicitly include citations to such documents and/or explicitly include the functionally corresponding structures which were incorporated by reference above.

Thus, for any invention element(s)/structure(s) corresponding to functional claim limitation(s), in the below claims, that are interpreted under 35 USC § 112(6) (post AIA 112(f)), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of implementing a vermiculture filtration system according to the present invention will be apparent to those skilled in the art. Various aspects of the invention have been described above by way of illustration, and the specific embodiments disclosed are not intended to limit the invention to the particular forms disclosed. The particular implementation of the vermiculture filtration system may vary depending upon the particular context or application. By way of example, and not limitation, the vermiculture filtration system described in the foregoing were principally directed to earthworm water filtering implementations; however, similar techniques may instead be applied to growing worms as fishing bait, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

Only those claims which employ the words “means for” or “steps for” are to be interpreted under 35 USC 112, sixth paragraph (pre AIA) or 35 USC 112(f) post-AIA. Otherwise, no limitations from the specification are to be read into any claims, unless those limitations are expressly included in the claims.

What is claimed is:

1. A method comprising the steps of:
transferring waste material to one or more containment and/or distribution tank(s), the waste material including animal waste, human waste, wastewater, contaminated liquid, industrial waste or sludge;
holding the waste water in said one or more containment and/or distribution tank(s);
transferring the waste water to one or more process tank(s) with a catch basin;
timing the transfer of the waste water from said one or more distribution tank(s) to said one or more process tank(s) to allow earthworms in the one or more process tank(s) to process the waste water;
draining the processed waste water to a sand filter with a catch basin;
polishing, with said sand filter, the processed waste water;
draining the processed and polished waste water with a UV filter; and
sterilizing the processed and polished waste water with said UV filter.

2. The method of claim 1, further comprising the steps of filtering animal liquid waste, human sewage waste, biological process plant waste or organic contaminated liquids with earthworms to significantly reduce greenhouse gas production, eliminate odor emissions, reduce nitrate contamination, and/or eliminate human indicator pathogens while producing organic soil amendment and usable water for use in agriculture and for reuse as wash down water.

3. The method of claim 1, further comprising the step of pulse time feeding the waste water to said one or more process tank(s), where the waste water feeds for a period of time then pauses for a period of time.

4. The method of claim 1, further comprising the steps of:
housing the earthworms in said one or more process tank(s); and
installing a hinged dump bottom in said process tank(s), to allow simplified or easy removal of castings.

5. The method of claim 4, further comprising the steps of:
installing a mature earthworm capture separation screen to allow simplified removal of mature earthworms; and
installing a juvenile earthworm separation screen for egg capsule and juvenile worm capture.

6. The method of claim 5, further comprising the step of applying a cellulose porous media to said earthworm capture screen to provide media for the adult worms to be shielded from light while entering said capture screen.

7. The method of claim 1, further comprising the steps of:
installing a wire mesh platform under each of said one or more process tank(s) to allow passage of processed liquid;
installing said catch basin under the wire mesh platform to capture the process liquid then direct it to said sand filter; and
holding at least one liquid distribution piping inside said catch basin for distributing the contaminated liquid.

8. The method of claim 1, further comprising the steps of installing a light under each catch basin to prevent earthworms from leaving the process tanks.

9. The method of claim 1, further comprising the steps of:
installing a process screen at an upper part of said sand filter to remove the top level of the sand to maintain polishing efficiency; and
installing an ultraviolet water purifier to assure no pathogens remain in the cleaned water.

10. The method of claim 1, wherein the contaminated liquid or waste water is a mix of solids and liquids and at least one of, human sewage waste, animal liquid waste including swine, beef, dairy, or chicken, and commercial biological process plant waste.

11. The method of claim 1, further comprising the steps of:
housing said one or more containment and/or distribution tank(s), one or more process tank(s), sand filter and UV filter inside a building to allow for temperature control and final scrubbing of process air;
installing a carbon filter and earthworm castings filter for filtration of noxious air odors;
installing a monitoring equipment for GHG reduction verification (Methane, Ammonia, CO2, Nox, and others), nitrate contamination reduction and odor level detection; and
installing an air conditioning to keep the temperature inside the building at an optimal level.

12. A system comprising:
means for holding waste material including wastewater, contaminated liquid, animal waste, human waste or sludge;
means for housing earthworms;
means for transferring the wastewater from said means for holding wastewater to said means for housing earthworms;
means for timing the transfer of the wastewater from said means for holding wastewater to said means for housing earthworms to allow earthworms in said earthworms housing means to process the waste water;
means for removing suspended matter/debris and/or floating/sinkable particles present in the wastewater;
means for draining the processed wastewater to said means for removing suspended matter/debris and/or floating/sinkable particles present in the wastewater;
means for sterilizing the wastewater with UV light; and
means for draining the processed and polished waste water to said means for sterilizing the wastewater with UV light.

13. A system comprising:
one or more containment and/or distribution tank(s) that is configured to be operable for holding wastewater or contaminated liquid;
one or more process tank(s) that is configured to be operable for housing earthworms;
one or more process tank catch basin(s) for each of said one or more process tank(s);
a timing valve, said timing valve is configured to control a volume of wastewater that is distributed to said one or more process tank(s);
a sand filter, said sand filter is operable for removing suspended matter/debris and/or floating/sinkable particles present in the wastewater;
a sand filter catch basin;
a UV filter;

14. The system of claim 13, further comprising one or more hinged dump bottom in said one or more process tank(s) that are configured to allow simplified or easy removal of earthworm castings.

15. The system of claim 14, further comprising:
a mature earthworm capture separation screen that is configured to allow a simplified removal of mature earthworms; and
a juvenile earthworm separation screen that is operable for egg capsule and juvenile worm capture.

16. The system of claim 13, further comprising:
a pallet rack; and
a wire mesh platform under each of said one or more process tank(s) to allow passage of processed liquid.

17. The system of claim 13, further comprising:
a process screen at an upper part of said sand filter to remove a top level of said sand filter to maintain polishing efficiency; and
an ultraviolet water purifier to assure no pathogens remain in the cleaned water.

18. The system of claim 13, further comprising an ultraviolet water purifier to assure no pathogens remain in the cleaned water.

19. The system of claim 15, wherein said separation screen comprise pivot holes to allow each half of said separation screen to be turned over in a bottom of each process tank.

20. The system of claim 13, wherein said sand filter comprises:
a sand filter tank;
a hinged bottom, said hinged bottom is configured to be operable for allowing water to be drained and sand to be dumped and refilled;
a hinge point, said hinge point engages said hinged bottom to said sand filter tank;
a screen, said screen is configured to be operable for separating a top layer from a bottom area of said sand filter; and
a hook for supporting said screen on a top portion of said sand filter.
